(19) United States
(12) Patent Application Publication
(10) Pub. No.: US 2004/0248291 A1
(43) Pub. Date: Dec. 9, 2004

(54) Method for culturing cells, cell culture carriers and cell culture apparatus

(75) Inventors: Akira Yamamoto, Tokyo (JP); Ken Sugo, Saitama (JP); Yae Kurosawa, Kanagawa (JP); Kikuka Kishiro, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 10/822,006

(22) Filed: Apr. 12, 2004

(30) Foreign Application Priority Data
Apr. 10, 2003 (JP) 2003-107143
Apr. 10, 2003 (JP) 2003-107144

(51) Int. Cl. C12N 5/02

(52) U.S. Cl. 435/325

(57) ABSTRACT

A cell culturing method comprises the steps of preparing a cell culture solution containing at least cells to be cultured and granular cell culture carriers; and applying a magnetic field to the cell culture solution so as to agitate the cell culture solution by the effect of the magnetic field, whereby the cells adhere to and grow on the surfaces of the cell culture carriers. Each of the carriers comprises a magnetic particle having a coating layer which is provided to cover at least a part of the surface of the magnetic particle so that the cells are allowed to adhere thereto, wherein the cell culture carriers are moved in the culture solution by the application of the magnetic field, thereby agitating the culture solution. Alternatively, the culture solution further contains magnetic particles, and the magnetic particles are moved in the culture solution by the application of the magnetic field, thereby agitating the culture solution.

### BACKGROUND OF THE INVENTION

1. Field of the Invention

The present invention relates to a method for culturing cells (cell culturing method), cell culture carriers and a cell culture apparatus.

2. Description of the Prior Art

In recent years, cell culture technology is used in various industrial and research fields such as cell tissue engineering, safety tests of drugs, production of proteins for treatment or diagnosis purposes, and the like.

Recently, in order to culture a large number of anchorage-dependent cells efficiently, cell culture is carried out by three-dimensional high-density culture (microcarrier culture) instead of plate culture which is commonly used. While in the plate culture a culture flask is used, in the microcarrier culture a number of bead-like carriers which serve as scaffolds for cells are employed.

In such microcarrier culture, various types of cell culture carriers (carriers for cell culture) are used.

In the meantime, in such microcarrier culture, it is important to agitate a culture solution sufficiently during the cell culture process, in order to suspend carriers uniformly so that nutrition can be equally supplied to each cell adhering to the carriers.

Heretofore, in such microcarrier culture, a spinner flask having a stirring bar equipped with a fin has been used, in which the spinner flask rotates the stirring bar to agitate a culture solution (see Japanese Patent Laid-open No. Hei 06-209761).

However, in the microcarrier culture using such a spinner flask, if the rotational speed of the stirring bar is too high, there is a case that the fin and carriers heavily come into collision so that the cells come off from the carriers or the cells are damaged. This makes it impossible for the cells to satisfactorily grow. On the other hand, if the rotational speed of the stirring bar is too low, the carriers are likely to settle down in the spinner flask so that the carriers are not uniformly suspended in a culture solution. In such a case, nutrition is not equally supplied to each cell so that a desired growth rate cannot be obtained. For these reasons, it is extremely important to rotate the stirring bar at an appropriate rotational speed.

However, a problem exists with such microcarrier culture using the spinner flask in that it is very difficult to rotate the stirring bar at an appropriate rotational speed. There is also a problem in that it is necessary to set culture conditions (the rotational speed of the stirring bar, and the like) according to the kind of cell to be used, the shape of the flask, and the like.

### SUMMARY OF THE INVENTION

It is therefore an object of the present invention to provide a method for culturing cells, cell culture carriers and a cell culture apparatus which are capable of agitating a culture solution uniformly and gently (mildly).

In order to achieve the above-mentioned object, a method for culturing cells. The method comprises the steps of: preparing a cell culture solution containing at least cells to be cultured and granular cell culture carriers to which the cells are allowed to adhere and grow thereon; and applying a magnetic field to the cell culture solution so as to agitate the cell culture solution by the effect of the magnetic field, whereby the cells adhere to and grow on the surfaces of the cell culture carriers.

According to the method described above, it is possible to agitate the culture solution uniformly and mildly without detachment of the cells from the cell culture carriers, thereby enabling the cells to grow efficiently.

In one embodiment of the present invention, each of the carriers may comprise a magnetic particle having a surface and a coating layer which is provided to cover at least a part of the surface of the magnetic particle so that the cells are allowed to adhere thereto, wherein the cell culture carriers are moved in the culture solution by the application of the magnetic field, thereby agitating the culture solution. According to this method, it is possible to agitate the culture solution by containing only the cells and the cell culture carriers in the culture solution.

In this embodiment, it is preferred that the intensity of the magnetic field applied to the cell culture solution is changed with the lapse of time, and/or the position of the magnetic field applied to the cell culture solution is also changed with the lapse of time. This makes it possible to agitate the culture solution more uniformly and gently.

Further, in this embodiment, it is preferred that a density of each of the cell culture carriers is in the range of 0.8 to 2.5 g/cm³. This makes it possible to agitate the culture solution satisfactorily.

Furthermore, in this embodiment, it is also preferred that when the average particle size of the cell culture carriers is defined as A μm and the maximum length of the cell allowed to adhere to the cell culture carrier is defined as B μm, A/B is 2 to 100. This makes it possible to sufficiently enlarge a surface area of the cell culture carrier as compared with the size of the cell, thus making it easy for the cells to adhere to and grow on the surfaces of the cell culture carriers.

Moreover, in this embodiment, it is also preferred that the average particle size of the cell culture carriers is in the rage of 50 to 500 μm.

Further, in this embodiment, it is also preferred that the coating layer is mainly made of a calcium phosphate-based compound. Since the calcium phosphate-based compound is biologically inert, there is less possibility that gives damage to cells.

In this case, it is preferred that the coating layer is formed from fine particles of the calcium phosphate-based compound wherein the particles being partially embedded in a surface area including and adjacent to the surface of the magnetic particle. This makes it possible to provide excellent adhesion between the coating layer and the magnetic particle.

Further, in this case, the coating layer may be colliding the porous particles of the calcium phosphate-based compound to the surface of the magnetic particle. This makes it possible to form the coating layer easily and reliably.

Further, in this embodiment, it is preferred that each of the magnetic particles is formed by compounding a resin material and a magnetic material. According to this method, it is possible to adjust a density (specific gravity) of the magnetic particle (consequently, the cell culture carrier) by setting compounding ratio (mixing ratio) between the resin material and the magnetic material appropriately. Further, the shape and size of the cell culture carrier can also be adjusted easily.

In another embodiment of the present invention, the culture solution may further contain magnetic particles, and the magnetic particles are moved in the culture solution by the application of the magnetic field, thereby agitating the culture solution. According to this method, it is possible to agitate the culture solution uniformly and gently by simply adding the magnetic particles into the culture solution in addition to the cells and the cell culture carriers.

In this embodiment, it is also preferred that the intensity of the magnetic field applied to the cell culture solution is changed with the lapse of time, and/or the position of the magnetic field applied to the cell culture solution is changed with the lapse of time.

Further, in this embodiment, it is preferred that each of the cell culture carriers comprises a base body made of a resin material and having a surface and a coating layer which is provided to cover at least a part of the surface of the base body so that the cells are allowed to adhere thereto.

In this case, it is preferred that the coating layer is mainly made of a calcium phosphate-based compound.

Further, in this case, it is also preferred that the coating layer is formed from fine particles of the calcium phosphate-based compound wherein the particles being partially embedded in a surface area including and adjacent to the surface of the base body.

Further, in this case, it is preferred that the particles of the calcium phosphate-based compound are formed from porous particles, and the coating layer is formed by colliding the porous particles to the surface of the magnetic particle.

Further, in this case, it is also preferred that a density of each of the cell culture carriers is in the range of 0.8 to 1.4 g/cm³.

Furthermore, in this embodiment, it is also preferred that when the average particle size of the cell culture carriers is defined as A μm and the maximum length of the cell that is allowed to adhere to the cell culture carrier is defined as B μm, A/B is 2 to 100.

In this case, it is also preferred that when the average particle size of the cell culture carriers is defined as A μm and the average particle size of the magnetic particles is defined as C μm, C/A is 0.02 to 10.

Further, in this case, it is also preferred that the average particle size of the cell culture carriers is in the rage of 50 to 500 μm.

Further, in this embodiment, it is preferred that each of the magnetic particles is formed by compounding a resin material and a magnetic material.

Further, in this embodiment, it is also preferred that a density of each of the magnetic particles is in the range of 0.8 to 2.5 g/cm³.

Furthermore, it is also preferred that the average particle size of the magnetic particles is in the range of 10 to 500 μm.

In one modification of this embodiment, each of the magnetic particles may further comprise a coating layer which covers at least a part of the surface of the magnetic powder so that the cells are allowed to adhere thereto.

In this case, it is preferred that the coating layer is mainly made of a calcium phosphate-based compound.

Further, it is also preferred that the coating layer is formed from fine particles of the calcium phosphate-based compound wherein the particles being partially embedded in a surface area including and adjacent to the surface of the magnetic particle.

In this case, it is preferred that the particles of the calcium phosphate-based compound are formed from porous particles, and the coating layer is formed by colliding the porous particles to the surface of the magnetic particle.

Further, in this embodiment, it is preferred that a mixing ratio of the magnetic particles and the cell culture carriers is in the range of 10:90 to 50:50 in a volume ratio.

Another aspect of the present invention is directed to cell culture carriers to which cells are allowed to adhere to and grow on surfaces thereof, wherein each of the carriers comprising a magnetic particle having a surface, and a coating layer which is provided to cover at least a part of the surface of the magnetic particle so that the cells are allowed to adhere thereto.

In the cell culture carriers, it is preferred that a density of the carrier is in the range of 0.8 to 1.4 g/cm³.

Further, it is also preferred that when the average particle size of the cell culture carriers is defined as A μm and the maximum length of the cell that is allowed to adhere to the cell culture carrier is defined as B μm, A/B is 2 to 100.

In this case, it is preferred that the particle size of the cell culture carriers is in the rage of 50 to 500 μm.

Further, in the cell culture carriers, it is preferred that the coating layer is mainly made of a calcium phosphate-based compound.

In this case, it is preferred that the coating layer is formed from fine particles of the calcium phosphate-based compound wherein the fine particles being partially embedded into the magnetic particle at the vicinity of the surface thereof.

Further, it is also preferred that the fine particles of the calcium phosphate-based compound are formed from porous particles, and the coating layer is formed by colliding the porous particles to the surface of the magnetic particle.

Further, it is also preferred that the magnetic particles are formed by compounding a resin material and a magnetic material.

Yet another aspect of the present invention is directed to a cell culture apparatus. The cell culture apparatus comprises a cell culture vessel for storing a cell culture solution containing at least cells to be cultured and granular cell culture carriers to which the cells are allowed to adhere and grow thereon, and at leas one magnetic field generator for applying a magnetic field to the culture solution to agitate the culture solution by the effect of the magnetic field.

According to the cell culture apparatus described above, it is possible to agitate the culture solution uniformly and mildly without detachment of the cells from the cell culture carriers, thereby enabling the cells to grow efficiently.

In this cell culture apparatus of the present invention, it is preferred that each of the carriers comprises a magnetic particle having a surface and a coating layer which is provided to cover at least a part of the surface of the magnetic particle so that the cells are allowed to adhere thereto, wherein the cell culture carriers are moved in the culture solution by the application of the magnetic field, thereby agitating the culture solution.

Further, it is also preferred that the culture solution further contains magnetic particles, and the magnetic particles are moved in the culture solution by the application of the magnetic field, thereby agitating the culture solution.

In the cell culture apparatus of the present invention, the magnetic field generator is constructed so that the intensity of the generated magnetic field is changed with the lapse of time, and/or the magnetic field generator is constructed so that the position of the generated magnetic field is changed with the lapse of time. This makes it possible to agitate the culture solution more uniformly and gently.

In the cell culture apparatus, it is also preferred that the magnetic field generator is arranged around the outer periphery of the cell culture vessel. Further, it is also preferred that the magnetic field generator is provided so as to come into contact with the culture solution. Furthermore, it is also preferred that the magnetic field generator is arrange in the vicinity of the liquid surface of the culture solution contained in the cell culture vessel. These arrangements make it possible to move the cell culture carriers or the magnetic particles widely in the up and down directions in the culture solution, thus enabling to agitate the culture solution more uniformly.

Further, in the cell culture apparatus of the present invention, it is also preferred that the at leas one magnetic field generator includes two or more magnetic field generators. This makes it possible for the cell culture carriers or the magnetic particles to move in the culture solution with desired and complicated patterns.

These and other objects, structures and results of the present invention will be apparent more clearly when the following detailed description of the preferred embodiments is considered taken in conjunction with the accompanying drawings.

### BRIEF DESCRIPTION OF THE DRAWINGS

FIG. 1 is a cross-sectional view of a cell culture carrier used in a cell culturing method of a first embodiment of the present invention.

FIG. 2 is a cross-sectional view of a cell culture carrier used in a cell culturing method of a second embodiment of the present invention.

FIG. 3 is a cross-sectional view of a magnetic particle used in the cell culturing method of the second embodiment.

FIG. 4 is a cross-sectional view of a modification of the magnetic particle used in the cell culturing method of the second embodiment.

FIG. 5 is a schematic perspective view of a cell culture apparatus of a first embodiment of the present invention.

FIGS. 6(a) to 6(c) is a timing chart which shows patterns of a magnetic field generated by a magnetic field generator of the cell culture apparatus of the first embodiment.

FIG. 7 is a schematic perspective view of a cell culture apparatus of a second embodiment of the present invention.

FIG. 8 is schematic perspective view of a cell culture apparatus of a third embodiment of the present invention.

FIG. 9 is a timing chart which shows patterns of a magnetic field generated by a magnetic field generator of the cell culture apparatus of the third embodiment.

### DETAILED DESCRIPTION OF THE INVENTION

The present invention is applied to spinner culture in which cells are allowed to grow in a suspending state in a culture solution (liquid medium) with the culture solution being agitated.

In such spinner culture, particularly, in a case where anchorage-dependent cells are cultured, the cells and cell culture carriers (carriers for cell culture) are suspended in a culture solution, and the cells are allowed to grow in a state that the cells adhere to the surfaces of the carriers.

A cell culturing method using such cell culture carriers is particularly referred to as microcarrier culture, and the present invention can be suitably used for the microcarrier culture.

Hereinafter, a cell culturing method (method for culturing cells), cell culture carriers and a cell culture apparatus according to the present invention will be described based on preferred embodiments where the present invention is applied to the microcarrier culture as described above.

First, a cell culturing method of a first embodiment of the present invention will be described. The feature of the first embodiment of the present invention is directed to a cell culturing method which comprises the steps of preparing a cell culture solution containing cells to be cultured and granular cell culture carriers having magnetic particles; and applying a magnetic field to the cell culture solution so as to move the cell culture carriers in the cell culture solution to agitate the cell culture solution by the effect of the applied magnetic field, whereby the cells adhere to the surfaces of the cell culture carriers and grow thereon.

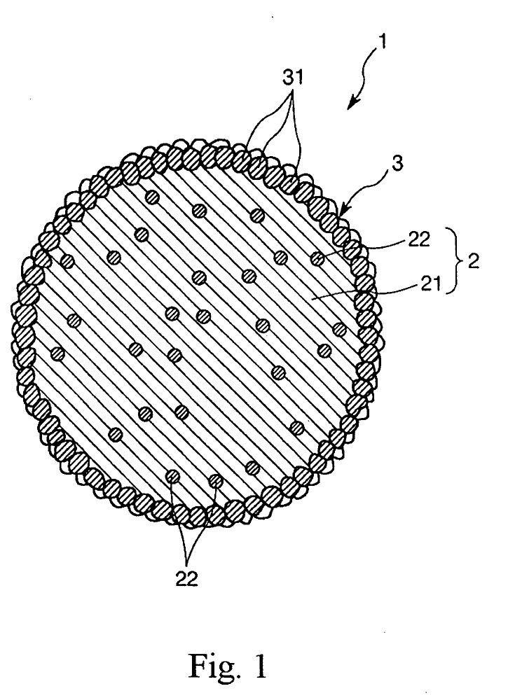

As described above, in this method, the cell culture carriers which are reactive with the magnetic field are used. FIG. 1 shows a cross-section of one of such cell culture carriers.

As shown in FIG. 1, a cell culture carrier 1 is comprised of a magnetic particle 2 and a coating layer 3 which covers a surface of the magnetic particle 2 so that cells are allowed to adhere thereto. The cell culture carrier 1 is moved in a culture solution when a magnetic filed is applied thereto, thereby agitating the culture solution uniformly and gently (mildly). Therefore, cells are easily to adhere onto the surface of each cell culture carrier 1, and nutrition is equally supplied to each cell. Therefore, this cell culture carrier 1 serves as a good scaffold for allowing the cells to grow.

Further, according to the method of the present invention, it is possible to prevent mechanical shock from being added to the cell culture carriers 1, which would be caused in the conventional method using a spinner flask due to collision of a fin and cell culture carriers, thus enabling to prevent the cells adhering to the cell culture carriers 1 from being fallen off from the surface thereof and also to prevent the cells from been damaged.

The magnetic particle 2 is a portion that constitutes a base of each cell culture carrier 1. The magnetic particle 2 may be formed of a magnetic material, but it is preferred that the magnetic particle is formed of a composite material which is obtained by compounding a resin material and a magnetic material. According to this structure, it is possible to adjust a density (specific gravity) of the magnetic material (that is, each cell culture carrier 1) easily by setting a compounding ratio (mixing ratio) of the resin material and the magnetic material appropriately. Further, there is another advantage in that the shape and size (average particle size or the like) of the cell culture carriers 1 can be easily adjusted.

As for the structure of the magnetic particle 2 (composite particle), it is preferred that, as shown in FIG. 1, a magnetic material (magnetic powder) 22 is dispersed in a base material 21 which is mainly made from the resin material. Such a magnetic particle 2 can be relatively easily manufactured by molding or granulating a resin material in a molten state to which the magnetic material has been mixed. In this regard, it is to be noted that in this magnetic particle 2 in the form of the composite particle, the magnetic material may be dispersed only in a portion of the base material 21 which is in the vicinity of the surface thereof.

Examples of the magnetic material 22 include a ferromagnetic alloy containing iron oxide, Fe, Ni, Co, or the like as a main component thereof, ferrite, barium ferrite, strontium ferrite, and the like. These magnetic materials may be used alone or in combination of two or more.

As for the resin material, various thermosetting resins and various thermoplastic resins can be used. Examples of the thermoplastic resins include polyamide, polyethylene, polypropylene, polystyrene, polyimide, an acrylic resin, and a thermoplastic polyurethane. Further, examples of the thermosetting resins include an epoxy resin, a phenol resin, a melamine resin, a urea resin, an unsaturated polyester, an alkyd resin, a thermosetting polyurethane, and ebonite. These resin materials may be used alone or in combination of two or more.

Further, the resin material may be colored with organic pigments, inorganic pigments, acid dyes, basic dyes, or the like.

The coating layer 3 is formed from a material to which cells can adhere. As for such a material, polystyrene, polyacrylamide, cellulose, dextran, and the like may be mentioned. However, a material containing a calcium phosphate-based compound as a main component thereof is particularly preferable. This is because the calcium phosphate-based compound is biologically inert, and thus there is less possibility that gives damage to cells.

In particular, when the coating layer 3 is formed using the calcium phosphate-based compound as a main material thereof, the coating layer 3 captures metal ions generated from the magnetic material 22 to prevent the elution of the metal ions into a culture solution. This makes it possible to prevent an adverse influence on cells. In this case, the coating layer 3 functions as an ion barrier layer.

The calcium phosphate-based compound is not particularly limited, and various compounds having a Ca/P ratio of 1.0 to 2.0 can be used. Examples of such a compound include Ca₁₀(PO₄)₆(OH)₂, Ca₁₀(PO₄)₆F₂, Ca₁₀(PO₄)₆Cl₂, Ca₃(PO₄)₂, Ca₂P₂)₇, Ca(PO₃)₂, and CaHPO₄. These compounds may be used alone or in combination of two or more.

Among them, as the calcium phosphate-based compound, one containing hydroxyapatite (Ca₁₀(PO₄)₆(OH)₂) as a main component is most suitable. Hydroxyapatite is used as a biomaterial, and thus cells can highly efficiently adhere thereto, and there is particularly less possibility that gives damage to the cells.

Further, in a case where fluorapatite (Ca₁₀(PO₄)₆F₂) is used, it is preferable that a fluorine content in the whole calcium phosphate-based compound is 5 wt % or less. By setting the fluorine content in the whole calcium phosphate-based compound to 5 wt % or less, it is possible to prevent or minimize the elution of fluorine from the coating layer 3 (that is, from the cell culture carrier 1). Therefore, damage to cells can be eliminated or minimized, and as a result, it is possible to prevent the growth efficiency of the cells from being lowered.

These calcium phosphate-based compounds can be synthesized by a known wet synthesis method, dry synthesis method or the like. In this case, the resulting calcium phosphate-based compound may contain a substance remaining as a result of synthesis (a raw material or the like) and/or a secondary reaction product produced in the course of synthesis.

In a case where the coating layer 3 is mainly formed of the calcium phosphate-based compound, the coating layer 3 may be formed by making the calcium phosphate-based compound to be adsorbed to the surface of the magnetic particle 2. However, it is preferred that, as shown in FIG. 1, the coating layer 3 is formed from fine particles 31 of the calcium phosphate-based compound (hereinafter simply referred to as “particles 31”) which are partially embedded in a surface area including and adjacent to the surface of the magnetic particle 2. This provides excellent adhesion between the coating layer 3 and the magnetic particle 2, thereby suitably preventing detachment of the coating layer 3 from the surface of the magnetic particle 2. Namely, it is possible to provide a cell culture carrier 1 having a sufficient strength.

In such a case, the coating layer 3 can be formed by, for example, colliding porous particles mainly formed of the calcium phosphate-based compound (hereinafter, simply referred to as “porous particles”) against the surface of the magnetic particle 2. According to such a method, it is possible to easily and reliably form the coating layer 3.

By colliding the porous particles against the surface of the magnetic particle 2, they are broken into fine particles 31 having a considerably small particle size when collided against the magnetic particle 2, and some of the particles 31 are partially embedded in the magnetic particle 2. When the particles 31 are partially embedded in the magnetic particle 2, the magnetic particle 2 captures the particles 31 due to its elastic force, thereby securing the particles 31 on the surface of the magnetic particle 2.

The porous particles are preferably produced by agglomerating primary particles of the calcium phosphate-based compound. By using such porous particles, it is possible to more reliably coat the surface of the magnetic particle 2 because such porous particles are more efficiently fragmented when collided against the magnetic particle 2.

The average particle size of the porous particles is not limited to any specific value, but is preferably 100 μm or less. If the average particle size of the porous particles exceeds 100 μm, there is a case that the velocity of the porous particle at the time of collision against the magnetic particle 2 becomes too low so that the porous particle is not efficiently fragmented.

A collision between the magnetic particles 2 and the porous particles can be carried out, for example, by using a commercially available hybridization machine in a dry condition. In such a case, the collision is carried out under the conditions that the mixing ratio between the magnetic particles 2 and the porous particles is about 400:1 to 50:1 in a weight ratio, and a temperature inside the machine is equal to or less than the softening temperature of the resin material which is used as a main material of the magnetic particle 2 (usually 80° C. or less), for example.

Such porous particles may also be produced, for example, in a known manner as follows.

Specifically, the porous particles may be produced by directly spray-drying a slurry containing crystalline particles (primary particles) of a calcium phosphate-based compound synthesized by a known wet method, to obtain granulated secondary particles. Alternatively, such secondary particles may be obtained by adding an additive such as a viscosity adjusting agent, particles of an organic compound or fibers which can be evaporated by heating, or the like to the slurry, and then spray-drying the slurry. It is to be noted that the obtained secondary particles may be sintered if necessary.

Since the thus obtained secondary particles are porous, such secondary particles can be directly used in forming the coating layer 3.

In a case where porous particles having higher porosity are preferable, such porous particles can be produced, for example, in the following manner.

First, a slurry in which the above-described secondary particles are suspended is prepared, and the slurry is formed into a block shape by wet molding, dry pressing, or the like. In this regard, it is to be noted that an organic compound which can be evaporated in the following sintering process to provide pores may be added to the slurry. The diameter of pores may be controlled by adjusting conditions such as a sintering temperature and the like instead of addition of such an organic compound. Then, the thus obtained block is sintered at a temperature within a range of about 400 to 1,300° C. If the sintering temperature is less than 400° C., there is a fear that the organic compound is not fully evaporated or the block is not satisfactorily sintered. On the other hand, if sintering is carried out at a high temperature exceeding 1,300° C., there is a fear that the resulting sintered body becomes excessively dense or the calcium phosphate-based compound is decomposed.

Thereafter, the thus sintered block is ground and then classified to obtain particles having a desired particle size.

The diameter of pores in the porous particle can be adjusted by, for example, appropriately setting the size of the primary particle, the viscosity of the slurry, the kind of additive, or the like.

The thus obtained porous particle preferably has a specific surface area of 10 m²/g or more, and a pore diameter of about 500 to 1,000 Å. A cell culture carrier 1 manufactured using porous particles satisfying these requirements enables cells to adhere to and grow on the surface thereof more efficiently.

It is to be noted that a method for forming the coating layer 3 (a method for manufacturing the cell culture carrier 1) is not limited to the method described above.

Further, the coating layer 3 may be either dense, e.g., non-porous, or porous.

Furthermore, the average thickness of the coating layers 3 is not limited to any specific value, but is preferably in the range of about 0.1 to 5 μm, more preferably in the range of about 0.5 to 2 μm. If the average thickness of the coating layers 3 is less than the above lower limit value, there is a fear that a part of the surface of the magnetic particle 2 is exposed in the cell culture carrier 1. On the other hand, if the average thickness of the coating layers 3 exceeds the above upper limit value, it becomes difficult to adjust the density of the cell culture carrier 1.

In this case, it is preferred that such a cell culture carrier 1 can be easily moved in a culture solution when a magnetic field is applied thereto and settled down or precipitated in the culture solution when the magnetic field is eliminated. By using such cell culture cells 1, it is possible to easily and reliably agitate the culture solution.

From this viewpoint, it is preferred that the density (specific gravity) of each cell culture carrier 1 is in the range of about 0.8 to 2.5 g/cm³, more preferably in the range of about 1.0 to 1.2 g/cm³. If the density of the cell culture carriers 1 is too small, it is difficult for the cell culture carriers 1 to be settle down in the culture solution when the magnetic field is eliminated. On the other hand, if the density of the cell culture carriers 1 is too large, it is necessary to apply a larger magnetic filed for moving the cell culture carriers 1 in the culture solution. In either cases, there is a fear that the culture solution can not be agitated sufficiently.

The size of the cell culture carrier culture 1 is not limited to any specific value, but the followings are preferable, for example.

That is, when the average particle size of the cell culture carriers 1 is defined as A (μm), and the maximum length of a cell which is allowed to adhere to the cell culture carrier 1 is defined as B (μm), A/B is preferably about 2 to 100, more preferably about 5 to 50. By setting A/B to a value within the above range, it is possible to sufficiently increase the surface area of each cell culture carrier 1 with respect to the size of the cell, thereby enabling the cells to adhere to and grow on the surfaces of the cell culture carriers 1 more easily.

Practically, the average particle size of the cell culture carriers 1 is preferably in the range of about 50 to 500 μm, more preferably in the range of about 100 to 300 μm. By setting the average particle size of the cell culture carriers 1 to a value within the above range, it is possible to further improve the effects described above.

In terms of enabling a larger number of cells to adhere to and grow on the surfaces of the cell culture carriers 1 described above, it is preferred that substantially entire surface of each magnetic particle 2 is covered with the coating layer 3, similarly to the present embodiment. However, the cell culture carrier 1 may have a structure in which a part of the surface of the magnetic particle 2 is covered with the coating layer 3, depending on the kind of cell to be cultured by the cell culture carrier 1, the kind of constituent material of the magnetic particle 2, or the like (that is, a structure in which a part of the surface of the magnetic particle 2 is exposed through gaps of the coating layer 3).

Next, a description will be made with regard to a cell culturing method of a second embodiment of the present invention, and cell culture carriers and magnetic particles used in the cell culturing method of the second embodiment.

The feature of the second embodiment is directed to a cell culturing method which comprises the steps of preparing a cell culture solution containing cells to be cultured, granular cell culture carriers and magnetic particles; and applying a magnetic field to the cell culture solution so as to move the magnetic particles in the cell culture solution to agitate the cell culture solution, thereby allowing the cells to adhere to and grow on the surfaces of the cell culture carriers.

That is, in a case where cell culture is carried out according to the cell culturing method of this second embodiment, first, the magnetic particles as well as the cells and the cell culture carriers are added to the culture solution. Then, the magnetic particles are moved in the culture solution by applying a magnetic field to the culture solution, so that the culture solution is agitated. In such a condition, the cells adhere to and grow on the surface of the cell culture carriers.

According to this embodiment, it is possible to agitate a culture solution uniformly and gently, and as a result, cells can grow efficiently.

Further, as will be described later in more details, such a method for culturing cells makes it possible to more uniformly agitate a culture solution by changing the intensity or position of a magnetic field to be applied to the culture solution with the lapse of time.

Hereinafter, each of the components of the cell culturing method of the second embodiment will be described one by one.

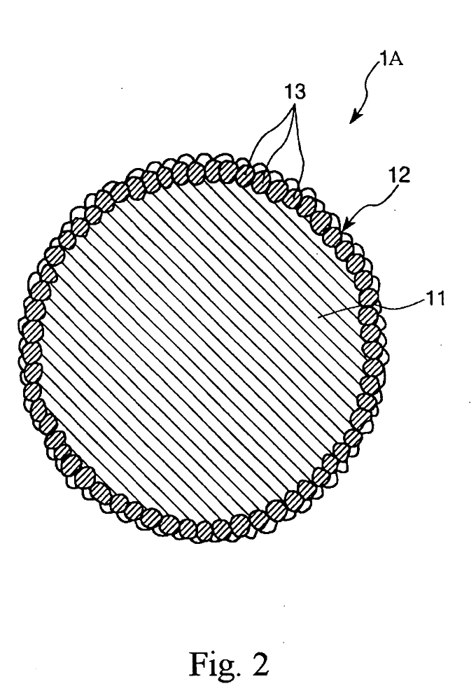

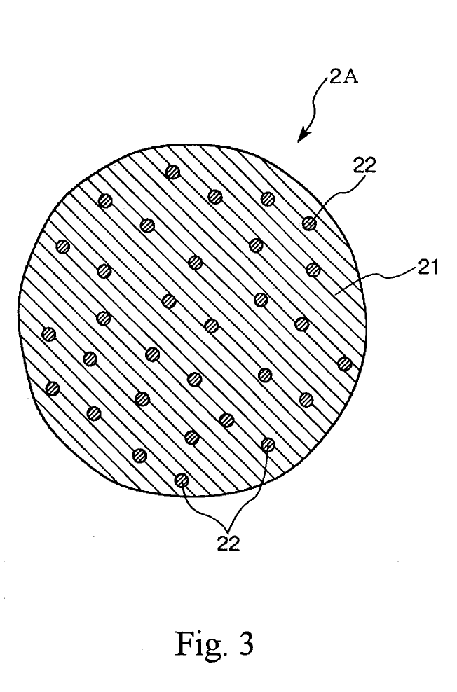

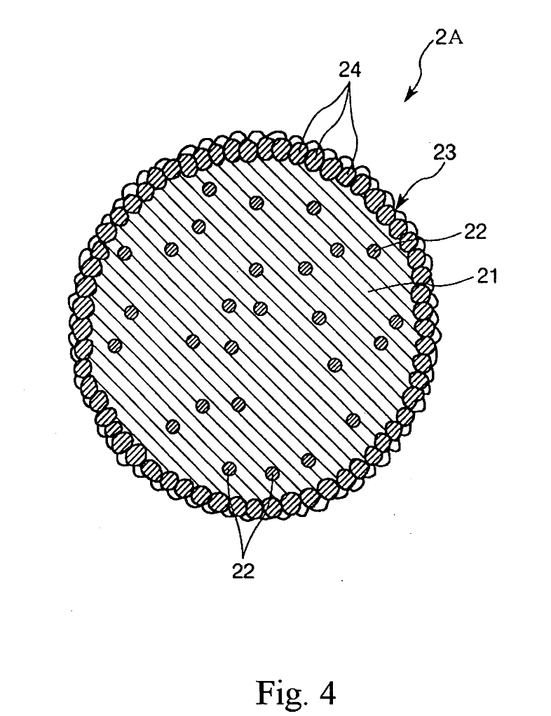

FIG. 2 is a cross-sectional view which shows the structure of the cell culture carrier used in the cell culturing method of the second embodiment, FIG. 3 is a cross-sectional view which shows the structure of the magnetic particle used in the cell culturing method of the second embodiment, and FIG. 4 is a cross-sectional view which shows a modification of the magnetic particle used in the cell culturing method of the second embodiment.

(Cell Culture Carrier 1A)

As is the same with the first embodiment, a cell culture carrier 1A serves as a scaffold for cell growth, and it is formed into a guranular or particulate shape (preferably in a substantially spherical granular shape).

As the cell culture carrier 1A, a carrier formed of a material containing as a major component thereof polystyrene, polyacrylamide, cellulose, or dextran, or the like may be used (which is usually used in microcarrier culture). However, in this second embodiment, cell culture carriers 1A each having a structure shown in FIG. 2 are used. Namely, each carrier 1A is comprised of a base body 11 and a coating layer 12 which is provided to cover the surface of the base body 11 so that cells are allowed to adhere to the coating layer 12.

The use of such a carrier having a structure shown in FIG. 2 as the cell culture carrier 1A makes it possible for the cell culture carrier 1A to suitably exhibit the function of allowing cells to adhere thereto and grow thereon, and makes it easy to adjust the shape, size (average particle size or the like), and physical properties (density and the like) of the cell culture carrier 1A. Hereinafter, a detailed description will be made with regard to this cell culture carrier 1A.

The base body 11 is preferably formed from a resin material. The use of such a base body makes it possible to further improve the effect described above.

As the resin material, various thermosetting resins and various thermoplastic resins can be used. Examples of the thermoplastic resins include polyamide, polyethylene, polypropylene, polystyrene, polyimide, an acrylic resin, and a thermoplastic polyurethane, and examples of the thermosetting resins include an epoxy resin, a phenol resin, a melamine resin, a urea resin, an unsaturated polyester, an alkyd resin, a thermosetting polyurethane, and ebonite. These resin materials may be used alone or in combination of two or more.

Further, the resin material may be colored with organic pigments, inorganic pigments, acid dyes, basic dyes, or the like.

As the constituent material of the coating layer 12, any material can be used as long as cells can adhere thereto, and in particular, one containing a calcium phosphate-based compound as a main material is suitable. The calcium phosphate-based compound is preferable since it is biologically inert and there is less possibility that cells are damaged.

The calcium phosphate-based compound is not particularly limited, and various compounds having a Ca/P ratio of 1.0 to 2.0 can be used. Examples of such a compound include Ca₁₀(PO₄)₆(OH)₂, Ca₁₀(PO₄)₆F₂, Ca₁₀(PO₄)₆Cl₂, Ca₃(PO₄)₂, Ca₂P₂A₇, Ca(PO₃)₂, and CaHPO₄. These compounds may be used alone or in combination of two or more.

Among them, as the calcium phosphate-based compound, one containing hydroxyapatite (Ca₁₀(PO₄)₆(OH)₂) as a main component is most suitable. Since hydroxyapatite is used as a biomaterial, cells can highly efficiently adhere thereto, and there is less possibility that the cells are damaged.

Further, in a case where fluorapatite (Ca₁₀(PO₄)₆F₂) is used, it is preferred that a fluorine content in the whole calcium phosphate-based compound is 5 wt % or less. By setting the fluorine content in the whole calcium phosphate-based compound to 5 wt % or less, it is possible to prevent or minimize the elution of fluorine from the coating layer 12 (that is, from the cell culture carrier 1A). Therefore, damage to cells can be eliminated or minimized, and as a result, the growth efficiency of the cells is prevented from being decreased.

These calcium phosphate-based compounds can be synthesized by a known wet synthesis method, dry synthesis method or the like. In this case, the resulting calcium phosphate-based compound may contain a substance remaining as a result of synthesis (a raw material or the like) and/or a secondary reaction product produced in the course of synthesis.

In a case where the coating layer 12 is formed of the calcium phosphate-based compound, the coating layer 12 may be formed by letting the calcium phosphate-based compound to adsorb to the surface of the base body 11. Preferably, as shown in FIG. 2, the coating layer 12 is formed from particles 13 of the calcium phosphate-based compound (hereinafter simply referred to as “particles 13”) which are partially embedded in a surface area including and adjacent to the surface of the base body 11. This provides excellent adhesion between the coating layer 12 and the base body 11, thereby suitably preventing detachment of the coating layer 12 from the surface of the base body 11. Namely, it is possible to obtain a cell culture carrier 1A having a sufficient strength.

In such a case, the coating layer 12 can be formed by, for example, colliding porous particles mainly formed of the calcium phosphate-based compound (hereinafter simply referred to as “porous particles”) against the surface of the base body 11. According to such a method, it is possible to easily and reliably form the coating layer 12.

By colliding the porous particles against the surface of the base body 11, they are broken into fine particles 13 having a considerably small particle size when collided against the base body 11, and the particles 13 are partially embedded in the base body 11. When the particles 13 are partially embedded in the base body 11, the base body 11 captures the particles 13 due to its elastic force, thereby securing the particles 13 on the base body 11.

The porous particles are preferably produced by agglomerating primary particles of the calcium phosphate-based compound. By using such porous particles, it is possible to more reliably coat the surface of the base body 11 because such porous particles are more efficiently fragmented when collided against the base body 11.

The average particle size of the porous particles is not limited to any specific value, but is preferably 100 μm or less. If the average particle size of the porous particles exceeds 100 μm, there is a case that the velocity of the porous particle at the time of collision against the base body 11 becomes too low so that the porous particle is not efficiently fragmented.

A collision between the base bodies 11 and the porous particles can be carried out, for example, by using a commercially available hybridization machine in a dry condition. As for conditions at this time, for example, the mixing ratio between the base bodies 11 and the porous particles is about 400:1 to 50:1 in a weight ratio, and a temperature inside the machine is equal to or less than the softening temperature of the resin material which is used as a main material of the base body 11 (usually 80° C. or less).

Such porous particles may also be produced, for example, in a known manner as follows.

Specifically, the porous particles may be produced by directly spray-drying a slurry containing crystalline particles (primary particles) of a calcium phosphate-based compound synthesized by a known wet method, to obtain granulated secondary particles. Alternatively, such secondary particles may be obtained by adding an additive such as a viscosity adjusting agent, particles of an organic compound or fibers which can be evaporated by heating, or the like to the slurry, and then spray-drying the slurry. It is to be noted that the obtained secondary particles may be sintered if necessary.

Since the thus obtained secondary particles are porous, such secondary particles can be directly used in forming the coating layer 12.

In a case where porous particles having higher porosity are preferable, such porous particles can be produced, for example, in the following manner.

First, a slurry in which the above-described secondary particles are suspended is prepared, and the slurry is formed into a block shape by wet molding, dry pressing, or the like. In this regard, it is to be noted that an organic compound which can be evaporated in the following sintering process to provide pores may be added to the slurry. The diameter of pores may be controlled by adjusting conditions such as a sintering temperature and the like instead of addition of such an organic compound. Then, the thus obtained block is sintered at a temperature within a range of about 400 to 1,300° C. If the sintering temperature is less than 400° C., there is a fear that the organic compound is not fully evaporated or the block is not satisfactorily sintered. On the other hand, if sintering is carried out at a high temperature exceeding 1,300° C., there is a fear that the resulting sintered body becomes excessively dense or the calcium phosphate-based compound is decomposed.

Thereafter, the thus sintered block is ground and then classified to obtain particles having a desired particle size.

The diameter of pores in the porous particle can be adjusted by, for example, appropriately setting the size of the primary particle, the viscosity of the slurry, the kind of additive, or the like.

The thus obtained porous particle preferably has a specific surface area of 10 m²/g or more, and a pore diameter of about 500 to 1,000 Å. A cell culture carrier 1A manufactured using porous particles satisfying these requirements enables cells to adhere to and grow on the surface thereof more efficiently.

It is to be noted that a method for forming the coating layer 12 (a method for manufacturing the cell culture carrier 1A) is not limited to the method described above.

Further, the coating layer 12 may be either dense, e.g., non-porous, or porous.

Furthermore, the average thickness of the coating layers 12 is not limited to any specific value, but is preferably in the range of about 0.1 to 5 μm, more preferably in the range of about 0.5 to 2 μm. If the average thickness of the coating layers 12 is less than the above lower limit value, there is a fear that a part of the surface of the base body 11 is exposed in the cell culture carrier 1A. On the other hand, if the average thickness of the coating layers 12 exceeds the above upper limit value, it becomes difficult to adjust the density of the cell culture carrier 1A.

Such a cell culture carrier 1A preferably has a density (specific gravity) close to that of water. Specifically, the density of the cell culture carrier 1A is preferably in the range of about 0.8 to 1.4 g/cm³, more preferably in the range of about 0.9 to 1.2 g/cm³. By setting the density of the cell culture carrier 1A to a value within the above range, it is possible to suspend the cell culture carriers 1A in a culture solution more uniformly.

The size of the cell culture carrier 1A is not limited to any specific value, but the followings are preferable, for example.

That is, when the average particle size of the cell culture carriers 1A is defined as A (μm), and the maximum length of a cell which is allowed to adhere to the cell culture carrier 1A is defined as B (μm), A/B is preferably about 2 to 100, more preferably about 5 to 50. By setting A/B to a value within the above range, it is possible to sufficiently increase the surface area of the cell culture carrier 1A with respect to the size of the cell, thereby enabling the cells to adhere to and grow on the surface of the cell culture carrier 1A more easily.

Further, when the average particle size of the cell culture carriers 1A is defined as A (μm), and the average particle size of magnetic particles 2A which will be described later is defined as C (μm), C/A is preferably about 0.02 to 10, more preferably about 0.3 to 3. By setting C/A to a value within the above range, it is possible to sufficiently agitate a culture solution by virtue of the movement of the magnetic particles 2A (see below), thereby enabling the carriers for cell culture 1A to be suspended in the culture solution more uniformly.

Specifically, the average particle size of the cell culture carriers 1A is preferably in the range of about 50 to 500 μm, more preferably in the range of about 100 to 300 μm. By setting the average particle size of the cell culture carriers 1A to a value within the above range, it is possible to further improve the effects described above.

In terms of enabling a larger number of cells to adhere to and grow on the surface of the cell culture carrier 1A described above, it is preferred that substantially all the surface of the base body 11 is covered with the coating layer 12, similarly to the present embodiment. However, the cell culture carrier 1A may have a structure in which a part of the surface of the base body 11 is covered with the coating layer 12, depending on the kind of cell which is allowed to adhere to the cell culture carrier 1A, the kind of constituent material of the base body 11, or the like (that is, a structure in which a part of the surface of the base body 11 is exposed through gaps of the coating layer 12).

(Magnetic Particle 2A)

The magnetic particle 2A can be moved in a culture solution due to the application of a magnetic field. When the magnetic particles 2A are moved throughout the culture solution, the culture solution is uniformly and mildly agitated, and as a result, the cell culture carriers 1A are uniformly suspended in the culture solution.

Therefore, it becomes easy for cells to adhere to the surface of the cell culture carriers 1A, and nutrition is equally supplied to the cells adhering to the cell culture carriers 1A. Further, it is possible to prevent mechanical shock from being added to the cell culture carriers 1, which would be caused in the conventional method using a spinner flask due to collision of a fin and cell culture carriers, thus enabling to prevent the cells adhering to the cell culture carriers 1A from being fallen off from the surface thereof and also to prevent the cells from been damaged. For this reason, according to the present invention, cells can grow more efficiently.

It is preferred that the magnetic particles 2A can be easily moved due to the application of a magnetic field and can settle down (precipitate) in a culture solution when the magnetic field is eliminated. This enables the culture solution to be more easily and reliably agitated.

From this viewpoint, the density (specific gravity) of the magnetic particle 2A is preferably in the range of about 0.8 to 2.5 g/cm³, more preferably in the range of about 1.2 to 1.9 g/cm³. If the density of the magnetic particle 2A is too small, it is difficult for the magnetic particles 2A to settle down in a culture solution when a magnetic field is eliminated. On the other hand, if the density of the magnetic particle 2A is too large, a great magnetic field is required to move the magnetic particles 2A in a culture solution. In either case, there is a fear that the culture solution cannot be sufficiently agitated.

The magnetic particle 2A may be formed of a magnetic material as a whole, but is preferably a composite material which is obtained by compounding a resin material and a magnetic material. In this case, by setting the compounding ratio (mixing ratio) between the resin material and the magnetic material appropriately, it is possible to easily adjust the density (specific gravity) of the magnetic particle 2A. In addition, there is an advantage that the shape and size (e.g., average particle size) of the magnetic particle 2A can be easily adjusted.

As shown in FIG. 3, the magnetic particle (composite particle) 2A preferably has a structure in which a magnetic material (magnetic powder) 22 is dispersed in a base body 21 mainly formed of the resin material. Such magnetic particles 2A can be manufactured relatively easily by, for example, forming the resin material in a molten state containing the magnetic material 22 into particles (granulating). In this regard, it is to be noted that the magnetic particle 2A in the form of the composite particle may have a structure in which the magnetic material 22 is dispersed only in the vicinity of the surface of the base body 21.

Examples of the magnetic material 22 include a ferromagnetic alloy containing iron oxide, Fe, Ni, Co, or the like as a main component thereof, ferrite, barium ferrite, strontium ferrite, and the like. These magnetic materials may be used alone or in combination of two or more.

As for the resin material, the same material as that mentioned above for the base body 11 of the cell culture carrier 1A can be used.

The average particle size of the magnetic particles 2A is preferably in the range of about 10 to 500 μm, more preferably in the range of about 100 to 300 μm. If the average particle size of the magnetic particles 2A is too small, a turbulent flow cannot be generated sufficiently in a culture solution. On the other hand, if the average particle size of the magnetic particles 2A is too large, a great magnetic field is required to move the magnetic particles 2A in a culture solution. In either case, there is a fear that the culture solution cannot be sufficiently agitated. In this regard, it is to be noted that if the average particle size of the magnetic particles 2A is too small, there is also a fear that the magnetic particles 2A are easily agglomerated together.

The amount of the magnetic particles 2A to be added to a culture solution is not limited to any specific value, but the magnetic particles 2A are preferably added so that the mixing ratio between the magnetic particles 2A and the cell culture carriers 1A may be in the range of about 10:90 to 50:50 (particularly, about 20:80 to 40:60) in a vol %. If the amount of the magnetic particles 2A to be added to a culture solution is too small, there is a fear that the culture solution cannot be sufficiently agitated. On the other hand, if the amount of the magnetic particles 2A to be added to a culture solution is too large, there is a fear that the frequency of a collision between the magnetic particles 2A and the cell culture carriers 1A increases so that cells come off from the cell culture carriers 1A.

Further, as shown in FIG. 4, such a magnetic particle 2A may have a modified structure in which at least a part of the surface (in FIG. 4, substantially all of the surface) of the magnetic particle 2A is covered with a coating layer 23 which enables cells to adhere thereto, for example. The use of such a magnetic particle 2A makes it possible for cells to adhere to and grow on the surfaces of the magnetic particles 2A, thereby further improving the growth efficiency of the cells.

The coating layer 23 preferably has the same structure as that of the coating layer 12 of the cell culture carrier 1A. That is, it is preferred that the coating layer 23 is mainly formed of the calcium phosphate-based compound. In addition, the coating layer 23 is preferably formed from fine particles 24 mainly made of the calcium phosphate-based compound which are partially embedded in a surface area including and adjacent to the surface of the magnetic particle 2A. In this case, it is preferred that the coating layer 23 is formed by colliding porous particles mainly formed of the calcium phosphate-based compound against the surface of the magnetic particle 2A.

In particular, when the coating layer 23 is formed using the calcium phosphate-based compound as a main material, the coating layer 23 captures metal ions generated from the magnetic material 22 to prevent the elution of the metal ions into a culture solution. This makes it possible to prevent an adverse influence on cells. In this case, the coating layer 23 functions as an ion barrier layer.

(Culture Solution)

In the cell culturing methods of the first and second embodiments of the present invention described above, the same culture solution may be used.

Specifically, a culture solution 140 is appropriately selected depending on the kind of cell to be used or the like, and is not limited to any specific one. Examples of a usable culture solution include Dulbecco's MEM, Nissui MEM, BME, MCDB-104 medium, and the like.

Further, the culture solution 140 may contain, for example, serum, serum protein such as albumin, and additives such as various vitamins, amino acid, and salts, if necessary.

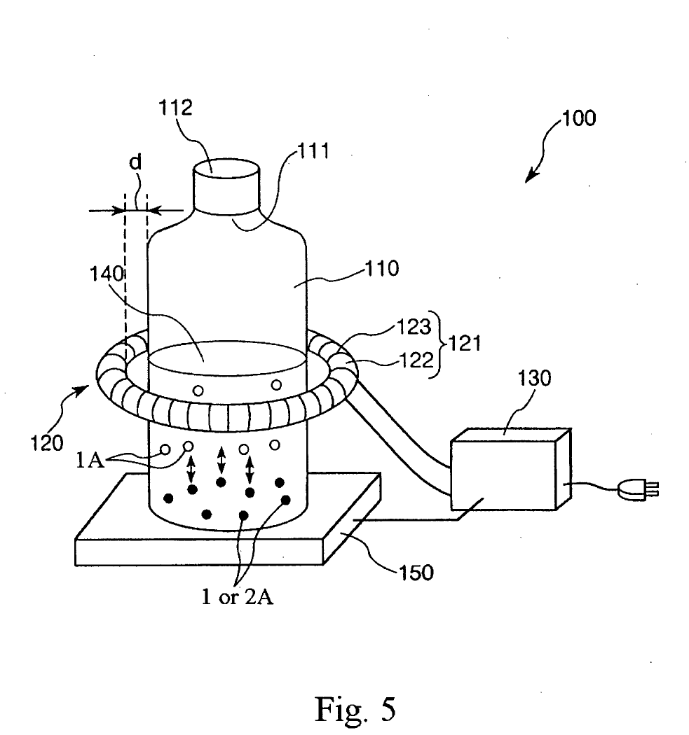

Next, with reference to FIGS. 5 to 9, a description will be made with regard to a cell culture apparatus which can be used for the cell culturing methods of the first and second embodiments described above.

### First Embodiment

First, a first embodiment of the cell culture apparatus of the present invention will be described.

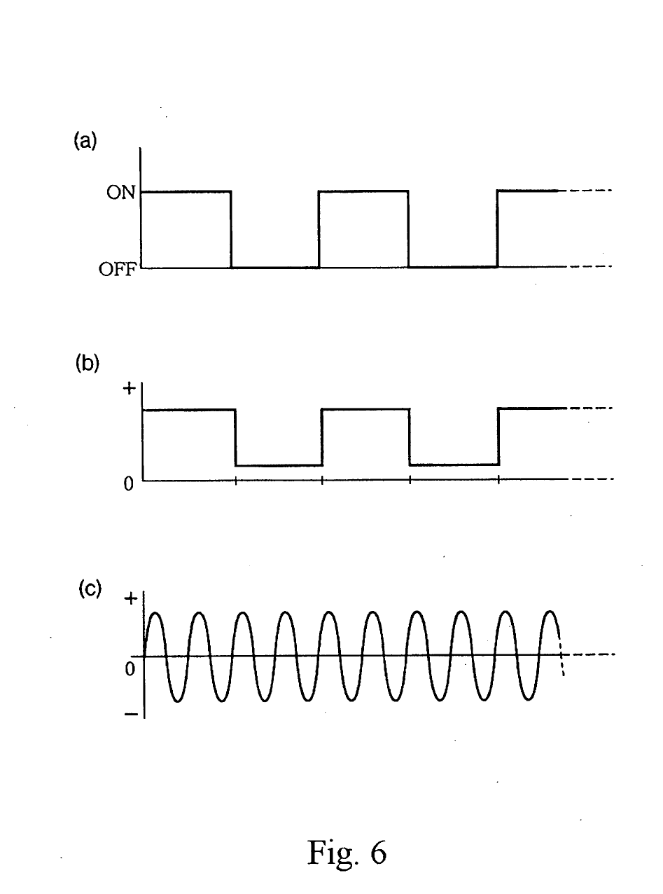

FIG. 5 is a schematic perspective which shows a cell culture apparatus of the first embodiment of the present invention, and FIG. 6 is a timing chart which shows the patterns of a magnetic field generated by a magnetic field generator of the cell culture apparatus.

A cell culture apparatus 100 shown in FIG. 5 has a culture vessel 110, a magnetic field generator 120, a controller 130, and a heating device 150. When the controller 130 is connected to a power source, electric power necessary to actuate each of the components of the cell culture apparatus 100 is supplied.

The culture vessel 110 is a component for receiving the culture solution 140, and has an opening 111, through which the culture solution 140 is fed and discharged, at the upper portion thereof. The opening 111 is closed with a plug 112 when necessary, to maintain air tightness within the culture vessel 110.

The shape, capacity, and the like of the culture vessel 110 are not particularly limited, and are appropriately determined depending on the kind of cell to be used, the kind of the culture solution 140, and the like.

The magnetic field generator 120 is a component for generating a magnetic field to move the cell culture carriers 1 used in the cell culturing method of the first embodiment or the magnetic particles 2A used in the cell culturing method of the second embodiment in the culture solution 140. The magnetic field generator 120 has an electromagnet 121 and a non-magnetic cover for accommodating the electromagnet 121 (not shown).

In the present embodiment, the electromagnet 121 is comprised of a toroidal metallic core material 122 and a conductor 123 spirally wound around the periphery of the core material 122. The passage of electric current through the conductor 123 generates a magnetic field in the vicinity of the conductor 123.

The non-magnetic cover has the function of protecting and fixing the electromagnet 121, and is made of various resin materials such as an acrylic-based resin and a silicone-based resin, for example.

There is provided the culture vessel 110 on the inside of such a magnetic field generator 120. In other words, the magnetic field generator 120 is provided around the periphery of the culture vessel 110. The magnetic field generator 120 is fixed and held by a fixing member (not shown). The position of the magnetic field generator 120 in the vertical direction of the culture vessel 110 is preferably set to a level in the vicinity of the surface of the culture solution 140 at the time when the culture solution 140 is stored (received) in the culture vessel 110. By setting the position of the magnetic field generator to such a level, it is possible to widely move the cell culture carriers 1 or the magnetic particles 2A in the vertical direction so that the culture solution 140 is agitated more uniformly.

The distance between the magnetic field generator 120 and the culture vessel 110 (which is represented by “d” in FIG. 5) is not particularly limited, but the magnetic field generator 120 and the culture vessel 110 are preferably disposed as close as possible. In particular, they are preferably disposed so as to come into (closely) contact with each other.

The magnetic field generator 120 is designed so that it can change the intensity of a magnetic field to be generated with the lapse of time. An example of the pattern of a magnetic field to be generated by the magnetic field generator 120 includes a pattern that a magnetic field is intermittently generated at regular intervals (see FIG. 6(a)). When a magnetic field is generated, the cell culture carriers 1 or the magnetic particles 2A are attracted to the side of the magnetic field generator 120 so that the cell culture carriers I or the magnetic particles 2A rise in the culture solution 140. In such a state, when the generation of the magnetic field is stopped, the cell culture carriers 1 or the magnetic particles 2A attracted to the side of the magnetic field generator 120 settle down under their self weight. By repeating such a vertical movement of the cell culture carriers 1 or the magnetic particles 2A, a turbulent flow is generated in the culture solution 140 so that the culture solution 140 is uniformly and gently agitated.

The maximum intensity (absolute value) of the magnetic field is appropriately determined depending on the density (specific gravity) of the cell culture carrier 1 or the magnetic particle 2A, the composition and volume of the culture solution 140, and the like, and is not limited to any specific value, but is preferably in the range of about 0.1 to 100 Wb/m², more preferably in the range of about 0.2 to 50 Wb/m². If the maximum intensity of the magnetic field (magnetic flux density) is too low, there is a fear that it becomes difficult to satisfactorily attract the cell culture carriers 1 or magnetic particles 2A to the side of the magnetic field generator 120 so that the culture solution 140 cannot be sufficiently agitated. On the other hand, if the maximum intensity of the magnetic field is increased over the above upper limit value, there is a fear that the magnetic field has an adverse influence on cells in addition to a waste of electric power.

The pattern of a magnetic field to be generated by the magnetic field generator 120 is not limited to the pattern that the magnetic field is intermittently generated at regular intervals (see FIG. 6(a)), and may be a pattern that the intensity of a magnetic field to be generated is increased and decreased at regular intervals (see FIG. 6(b)), a pattern that the intensity, direction, and the like of a magnetic field to be generated is continuously changed (see FIG. 6(c)), or the like, for example. These patterns may be optionally combined.

The controller 130 has the function of changing the conditions (e.g., kind, amount, direction, time, frequency, and the like) of electric current supplied from a power source. The controller 130 converts electric current supplied from a power source into electric current satisfying predetermined conditions (electric current having a predetermined pattern), and supplies the converted electric current to the electromagnet 121 (magnetic field generator 120). For example, in a case where the magnetic field generator 120 generates a magnetic field intermittently, the controller 130 converts an alternating current supplied from a power source into a pulsed current, and supplies the pulsed current to the electromagnet 121.

Further, the heating device 150 is electrically connected to the controller 130. The heating device 150 incorporates, for example, a heater, a peltier element, or the like, and heats the culture solution 140 under the control of the controller 130.

Next, an operation of the cell culture apparatus 100 (that is, the cell culturing method using the apparatus) will be described.

<1> First, the cell culture carriers 1 (in the case of the cell culturing method of the first embodiment) or the cell culture carriers 1A and the magnetic particles 2A (in the case of the cell culturing method of the second embodiment) are subjected to a sterilization treatment, respectively. This makes it possible to decrease the number of microorganisms or molds present on the surface of the cell culture carriers 1 or the cell culture carriers 1A and the magnetic particles 2A, or to fully kill the microorganisms or the molds. Therefore, a possibility that the microorganisms or the molds cause damage to cells is reduced or eliminated, enabling the cells to grow more efficiently.

In the sterilization treatment, a method in which the cell culture carriers 1 or the cell culture carriers 1A and the magnetic particles 2A are brought into contact with a sterilizing solution, autoclave sterilization, gaseous sterilization, radiation sterilization, or the like can be employed, for example. Among them, the method in which the cell culture carriers 1 or the cell culture carriers 1A and the magnetic particles 2A are brought into contact with a sterilizing solution is suitable. According to such a method, it is possible to more efficiently sterilize a large number of the cell culture carriers 1 or the cell culture carriers 1A and magnetic particles 2A.

In a case where a sterilizing solution is used, the cell culture carriers 1 or the cell culture carriers 1A and the magnetic particles 2A are washed after a sterilization treatment to remove the sterilizing solution adhering to the surface of the cell culture carriers 1 or the cell culture carriers 1A and the magnetic particles 2A.

<2> Next, the cell culture carriers 1 or the cell culture carriers 1A and the magnetic particles 2A after the completion of the above process <1>, and cells (which are allowed to adhere to the carriers 1A and the magnetic particles 2A) are added to or mixed with the culture solution 140, and the thus obtained culture solution 140 is stored (received) in the culture vessel 110 of the cell culture apparatus 100.

Here, for example, a shuttle vector (vector) containing a gene encoding a target protein has been previously introduced into each cell.

Examples of the cell include an animal cell, a plant cell, a bacterium, and a virus. Among them, an animal cell is particularly suitable. The use of an animal cell as such a cell makes it possible to apply the present invention in wider technical fields. In addition, in a case where protein is produced, one having a more complex structure (e.g., glycoprotein) can be suitably produced.

<3> Next, as has been described above, when the cell culture apparatus 100 is actuated, the magnetic field generator 120 applies a magnetic field having a predetermined pattern to the culture solution 140. By doing so, the cell culture carriers 1 or the cell culture carriers 1A and the magnetic particles 2A are moved in the culture solution 140, and the culture solution 140 is then agitated by virtue of the movement of the cell culture carriers 1 or the cell culture carriers 1A and the magnetic particles 2A so that the cell culture carriers 1 or the cell culture carriers 1A and the magnetic particles 2A are uniformly suspended in the culture solution 140.

At this time, the culture solution 140 is heated by the heating device 150. The temperature of the culture solution 140 is appropriately determined depending on the kind of cell to be cultured and the like, and is not limited to any specific value. In usual, the temperature is in the range of about 4 to 40° C., and is preferably in the range of about 25 to 37° C.

In such a condition, the cells adhere to and grow on the surface of the cell culture carriers 1 or the cell culture carriers 1A and the magnetic particles 2A in the culture solution 140. In particular, since the culture solution 140 is uniformly and mildly agitated by virtue of the movement of the magnetic particles 2, the cells can be highly efficiently cultured.

Then, the grown cells produce a target protein. The protein is discharged into the culture solution 140 or accumulated in the cells, for example.

At this time, the cell culture may be carried out with supplying a gas containing an oxygen gas if necessary.

<4> Next, the produced protein is collected. In a case where the protein discharged into the culture solution 140 is collected, the protein can be collected as follows, for example.

Specifically, agitation of the culture solution 140 is stopped in the above process <3>, and then a supernatant is collected after the carriers for cell culture carriers 1 or the cell culture carriers 1A and the magnetic particles 2A settle down in the culture solution 140. Alternatively, the culture solution 140 may be filtered to collect the resulting filtrate. Then, the collected solution (supernatant or filtrate) is treated (e.g., chromatography), thereby enabling the target protein to be easily collected.

### Second Embodiment

Next, a cell culture apparatus of a second embodiment of the present invention will be described.

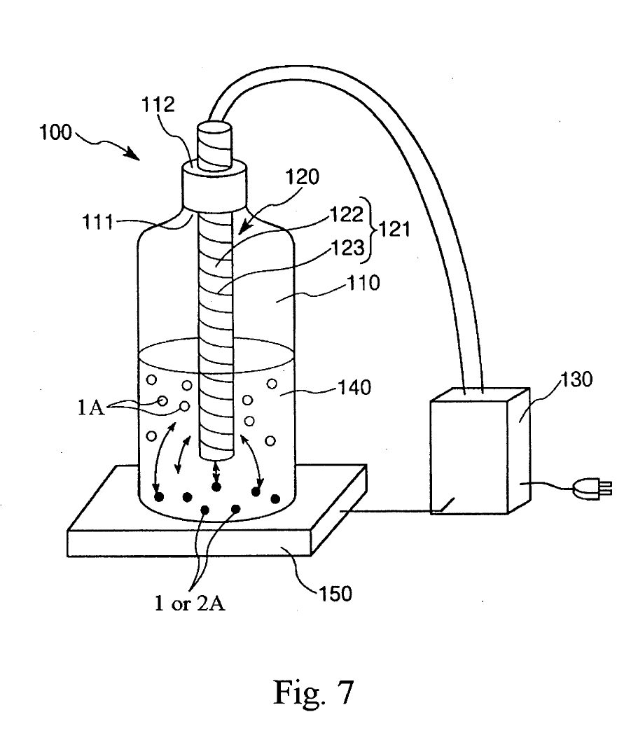

FIG. 7 is a schematic perspective view which shows the cell culture apparatus of the second embodiment of the present invention.

Hereinafter, the second embodiment will be described by focusing the difference between the first and second embodiments, and therefore a description of overlapping points will be omitted.

The cell culture apparatus 100 shown in FIG. 7 and the cell culture apparatus 100 of the first embodiment are the same except for the structure of the magnetic field generator 120.

Specifically, the magnetic field generator 120 of the second embodiment has the electromagnet 121 obtained by spirally winding the conductor 123 around the periphery of a straight (cylindrical) core material 122. The magnetic field generator 120 is preferably covered with a waterproof cover mainly formed of a material having no influence on a magnetic field.

The magnetic field generator 120 is fixed (secured) with it passing through the plug 112 to be attached to the culture vessel 110. In the present embodiment, the magnetic field generator 120 is disposed so as to come into contact with the culture solution 140 when cell culture is carried out.

The pattern of a magnetic field to be generated by the magnetic field generator 120 may be any one of the above-described patterns shown in FIG. 6, for example.

The cell culture apparatus 100 of the second embodiment can provide the same function and effect as those of the first embodiment.

### Third Embodiment

Next, a cell culture apparatus of a third embodiment of the present invention will be described.

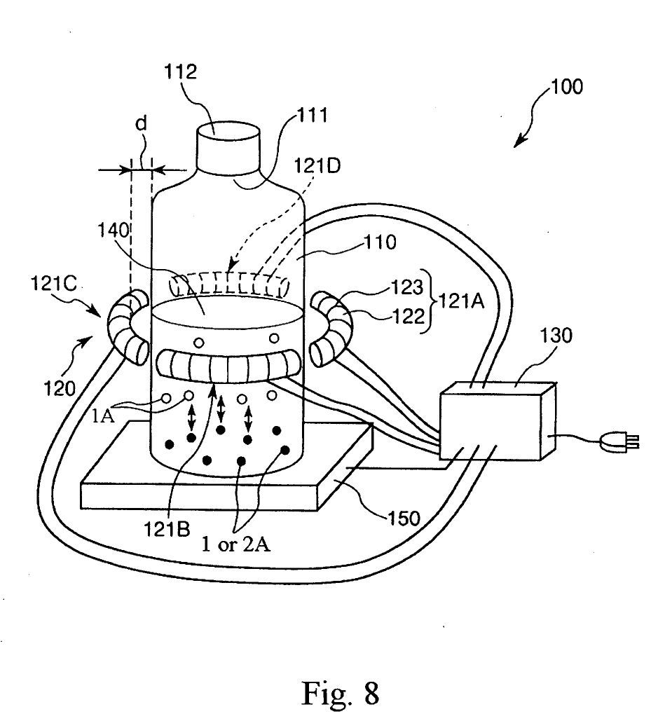

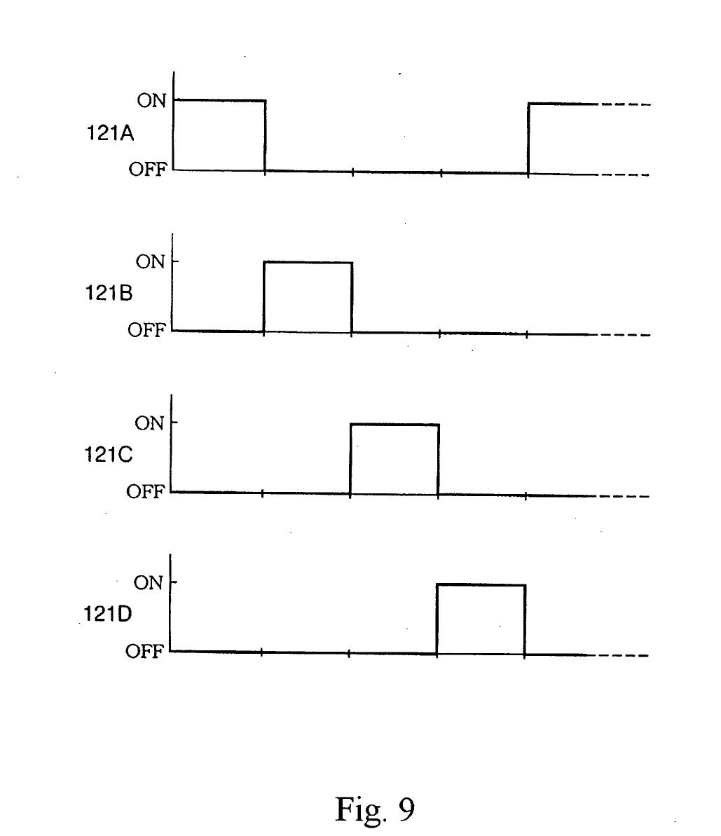

FIG. 8 is a schematic perspective view which shows the cell culture apparatus of the third embodiment of the present invention, and FIG. 9 is a timing chart which shows patterns of a magnetic field to be generated by the magnetic field generator.

Hereinafter, the third embodiment will be described by focusing the difference between the first and third embodiments, and therefore a description of overlapping points will be omitted.

The cell culture apparatus 100 shown in FIG. 8 and the cell culture apparatus 100 of the first embodiment are the same except for the structure of the magnetic field generator 120.

Specifically, the magnetic field generator 120 of the third embodiment has four (plural) electromagnets 121A to 121D.

The electromagnets 121A to 121D are spaced at substantially even intervals along the circumferential direction of the culture vessel 110.

With such a structure, successive switching among the electromagnets 121A to 121D to be electrified, that is, a change in the position of a magnetic field to be generated with the lapse of time makes it possible to further complicate the pattern of movement of the cell culture carriers 1 and the magnetic particles 2A in the culture solution 140. Therefore, the cell culture carriers 1 or the cell culture carrier 1A and the magnetic particles 2A are more uniformly suspended in the culture solution 140, and as a result, cells can grow more efficiently.

The pattern of a magnetic field to be generated by each of the electromagnets 121A to 121D (switching among the electromagnets 121A to 121D to be electrified) may be the pattern shown in FIG. 9, for example. Specifically, while one of the electromagnets generates a magnetic field, other electromagnets generate no magnetic field, and switching is carried out among the electromagnets to generate a magnetic field successively (synchronously). This makes it possible to move the cell culture carriers 1A or the magnetic particles 2A along the inner surface of the culture vessel 110.

It is to be noted that the pattern of a magnetic field to be generated by the electromagnets 121A to 121D is not limited to the pattern shown in Fin. 9, and may be a pattern obtained by optionally combining the patterns shown in FIG. 6, for example.

In the electromagnets 121A to 121D, for example, the winding number of the conductor 123, the overall shape, and the size thereof may be the same or different from each other.

The cell culture apparatus 100 of the third embodiment can provide the same function and effect as those of the first embodiment.

It should be noted that the present invention is not limited to the cell culture apparatuses described above, and so long as the same functions are achieved, it is possible to make various changes and additions to each portion thereof. In addition, two or more of the above-described embodiments may be optionally combined.

In each of the embodiments described above, the magnetic field generator is fixed. However, in the present invention, the magnetic field generator and the culture vessel may be provided so that they can be relatively moved to change the position of a magnetic field to be applied to a culture solution with the lapse of time. For example, the magnetic field generator may be moved in the vertical direction or horizontal direction with respect to the culture vessel, the magnetic field generator may be moved so as to get close to and get away from the culture vessel, and the magnetic field generator may be moved along the circumferential direction of the culture vessel. These movements of the magnetic field generator may be combined.

Further, in the present invention, the magnetic field generator may change the intensity of a magnetic field to be applied to a culture solution with the lapse of time under the pattern as described above while being relatively moved with respect to the culture vessel to change the position of the magnetic field to be applied to the culture solution with the lapse of time.

Furthermore, in each of the embodiments, the magnetic field generator has the electromagnet, but permanent magnets may be used instead of the electromagnet. In such a case, the magnetic field generator is further provided with a permanent magnet moving mechanism for moving permanent magnets with respect the culture vessel so that the cell culture carriers and/or the magnetic particles can be moved in the culture solution by the movement of the permanent magnets. In this case, the permanent magnets may be moved in various direction such as up and down directions, right and left directions, oblique directions and circumferential directions and any arbitral combinations of these directions.

Furthermore, in the cell culturing methods of the first and second embodiments described above, other cell culture carriers may be used together with the cell culture carriers mentioned above. As for such other cell culture carriers, carriers made of a material containing as a major component thereof polystyrene, polyacrylamide, cellulose, dextran, and the like, and carriers each comprised of a base body mainly made of a resin material and a coating layer which covers the surface of the base body and is made of a material to which cells can adhere thereto.

### EXAMPLES

Next, actual examples of the cell culturing methods of the first and second embodiments according to the present invention will be described.

### Cell Culturing Method of First Embodiment

1. Preparation of Cell Culture Carriers

(Cell Culture Carriers I-A)

First, 50 g of nylon particles (base bodies) having an average particle size of 150 μm and a density of 1.90 g/cm³, and 0.25 g of hydroxyapatite particles (porous particles obtained by agglomerating primary particles) having a Ca/P ratio of 1.67 and an average particle size of 10 μm were prepared.

The hydroxyapatite particle had a specific surface area of 45 m²/g, and a pore diameter of 600 Å.

Next, these nylon particles and hydroxyapatite particles were fed into a NARA HYBRIDIZATION SYSTEM NHS-1 (manufactured by Nara Machinery Co., Ltd. and having a rated power of 5.5 kW, and a rated current of 23 A), and the system was operated at 6,400 rpm at 32 to 50° C. for 5 minutes. In this way, cell culture carriers I-A coated with hydroxyapatite (each having a structure shown in FIG. 1) were obtained.

The thus obtained cell culture carrier I-A had an average particle size of 151 μm (the average thickness of coating layer of hydroxyapatite was 1 μm) and a density of 1.92 g/cm³.

(Cell Culture Carriers I-B)

First, 50 g of nylon particles (base bodies) having an average particle size of 150 μm and a density of 1.02 g/cm³, and 0.25 g of hydroxyapatite particles (porous particles obtained by agglomerating primary particles) having a Ca/P ratio of 1.67 and an average particle size of 10 μm were prepared.

The hydroxyapatite particle had a specific surface area of 45 m²/g, and a pore diameter of 600 Å.

Next, these nylon particles and hydroxyapatite particles were fed into a NARA HYBRIDIZATION SYSTEM NHS-1 (manufactured by Nara Machinery Co., Ltd. and having a rated power of 5.5 kW, and a rated current of 23 A), and the system was operated at 6,400 rpm at 32 to 50° C. for 5 minutes. In this way, cell culture carriers I-B coated with hydroxyapatite (each having a structure shown in FIG. 1) were obtained.

The thus obtained cell culture carrier I-B had an average particle size of 151 μm (the average thickness of coating layer of hydroxyapatite was 1 μm) and a density of 1.03 g/cm³.

(Cell Culture Carriers I-C)

Dextran particles having an average particle size of 200 μm and a density of 1.03 g/cm³ (manufactured by Pharmacia) were prepared as cell culture carriers I-C.

2. Culture of Cells

2-1 Culture of Cells Derived from Human Osteosarcoma (HOS)

This cell derived from human osteosarcoma is a cell having a maximum length of about 20 μm.

### Example Ia-1

1.5 g of the cell culture carriers I-A and 30 mL of a suspension containing 2×10⁵ cells derived from human osteosarcoma (HOS) per milliliter (/mL) were added to 100 mL of Nissui MEM (culture solution). It is to be noted that 10 vol % of bovine fetal serum was added to the Nissui MEM.

This culture solution was received in a culture vessel (which is a heatproof glass jar manufactured by IWAKI-PYREX) of a cell culture apparatus as shown in FIG. 5, and the cells were cultured. As for culture conditions, the pattern of a generated magnetic field was a pattern shown in FIG. 6(a), a pulse interval was 2 seconds, the temperature of the culture solution was 37° C., and a cultivation period was 5 days.

### Example Ia-2

Cell culture was carried out in the same manner as in Example Ia-1 except that the pulse interval was changed to 10 seconds.

### Example Ia-3

Cell culture was carried out in the same manner as in Example Ia-1 except that 1.5 g of the cell culture carriers I-A were replaced with 0.6 g of the cell culture carriers I-A and 0.8 g of the cell culture carriers I-B.

### Example Ia-4

Cell culture was carried out in the same manner as in Example Ia-1 except that 1.5 g of the cell culture carriers I-A were replaced with 0.6 g of the cell culture carriers I-A and 0.8 g of the cell culture carriers I-C.

### Comparative Example Ia-1

1.5 g of the cell culture carriers I-B and 30 mL of a suspension containing 2×10⁵ cells derived from human osteosarcoma (HOS) per milliliter (/mL) were added to 100 mL of Nissui MEM (culture solution). It is to be noted that 10 vol % of bovine fetal serum was added to the Nissui MEM.

This culture solution was received in a spinner flask (manufactured by Shibata Scientific Technology Ltd.), and the cells were cultured. Culture conditions were a rotational speed of stirring bar of 30 rpm, a temperature of culture solution of 37° C., and a cultivation period of 5 days.

### Comparative Example Ia-2

Cell culture was carried out in the same manner as in Comparative Example Ia-1 except that the rotational speed of the stirring bar was changed to 60 rpm.

### Comparative Example Ia-3

Cell culture was carried out in the same manner as in Comparative Example Ia-1 except that the cell culture carriers I-B were replaced with the cell culture carriers I-C.

2-2 Culture of Cells Derived from Monkey Kidney (Vero)

This cell derived from a monkey kidney is a cell having a maximum length of about 20 μm.

### Examples Ib-1 to Ib-4 and Comparative Examples Ib-1 to Ib-3

Cell culture was carried out in the same manner as in each of Examples Ia-1 to Ia-4 and Comparative Examples Ia-1 to Ia-3 except that the cells derived from human osteosarcoma were replaced with the cells derived from a monkey kidney.

2-3 Culture of Cells Derived from Mosquito (C6/36)

This cell derived from a mosquito is a cell having a maximum length of about 20 μm.

### Examples Ic-1 to Ic-4 and Comparative Examples Ic-1 to Ic-3

Cell culture was carried out in the same manner as in each of Examples Ia-1 to Ia-4 and Comparative Examples Ia-1 to Ia-3 except that the cells derived from human osteosarcoma were replaced with the cells derived from a mosquito.

3. Evaluation

In each of Examples and Comparative Examples, a predetermined amount of the culture solution was sampled after 3 hours, 1 day, 3 days and 5 days from the beginning of cultivation (beginning of agitation), and then the number of the cells adhering to the surface of the cell culture carriers (15 mg) was counted. The number of the cells was counted by subjecting the cells treated with EDTA or trypsin to trypanblue staining.

The results are shown in Tables 1 to 3.

TABLE 1
(Cells Derived from Human Osteosarcoma (HOS))

| | Cell | Number of cells (×10⁵ cells/mL) | | | |
|---|---|---|---|---|---|
| | Culture | After 3 | After 1 | After 3 | After 5 |
| | Carriers | hours | day | days | days |
| Ex. Ia-1 | I-A | 0.9 | 4.0 | 9.8 | 9.9 |
| Ex. Ia-2 | I-A | 0.9 | 4.2 | 9.9 | 10.1 |
| Ex. Ia-3 | I-A + I-B | 1.0 | 4.5 | 10.1 | 11.0 |
| Ex. Ia-4 | I-A + I-C | 1.0 | 4.8 | 10.2 | 10.8 |
| Comp. | I-B | 0.6 | 3.9 | 5.5 | 6.1 |
| Ex. Ia-1 | | | | | |
| Comp. | I-B | 0.4 | 0.6 | 0.5 | 0.3 |
| Ex. Ia-2 | | | | | |
| Comp. | I-C | 0.9 | 4.2 | 5.8 | 6.5 |
| Ex. Ia-3 | | | | | |

TABLE 2
(Cells Derived from Monkey Kidney (Vero))

| | Carriers | Number of cells (×10⁵ cells/mL) | | | |
|---|---|---|---|---|---|
| | for cell | After 3 | After 1 | After 3 | After 5 |
| | culture | hours | day | days | days |
| Ex. Ib-1 | I-A | 1.0 | 2.9 | 6.0 | 9.8 |
| Ex. Ib-2 | I-A | 0.9 | 3.0 | 6.0 | 9.9 |
| Ex. Ib-3 | I-A + I-B | 1.0 | 3.5 | 6.5 | 11.5 |
| Ex. Ib-4 | I-A + I-C | 1.0 | 3.4 | 7.0 | 11.1 |
| Comp. | I-B | 0.6 | 3.1 | 5.5 | 6.5 |
| Ex. Ib-1 | | | | | |
| Comp. | I-B | 0.5 | 0.4 | 0.1 | 0.1 |
| Ex. Ib-2 | | | | | |
| Comp. | I-C | 0.9 | 3.0 | 5.8 | 7.0 |
| Ex. Ib-3 | | | | | |

TABLE 3
(Cells Derived from Mosquito (C6/36))

| | Cell | Number of cells (×10⁵ cells/mL) | | | |
|---|---|---|---|---|---|
| | Culture | After 3 | After 1 | After 3 | After 5 |
| | Carriers | hours | day | days | days |
| Ex. Ic-1 | I-A | 0.8 | 2.5 | 5.6 | 8.3 |
| Ex. Ic-2 | I-A | 0.6 | 3.2 | 5.8 | 8.5 |
| Ex. Ic-3 | I-A + I-B | 0.9 | 4.2 | 8.0 | 9.2 |
| Ex. Ic-4 | I-A + I-C | 1.0 | 4.5 | 8.3 | 9.8 |
| Comp. | I-B | 0.4 | 2.5 | 4.8 | 5.2 |
| Ex. Ic-1 | | | | | |
| Comp. | I-B | 0.1 | 0.08 | 0.04 | 0.01 |
| Ex. Ic-2 | | | | | |
| Comp. | I-C | 0.6 | 2.5 | 5.0 | 5.9 |
| Ex. Ic-3 | | | | | |

As shown in each of the tables, it has become apparent that all Examples (present invention) exhibit higher efficiency of cell culture as compared with Comparative Examples irrespective of the kind of cell.

Further, even in a case where the pulse interval of generation pattern of a magnetic field was changed, Examples (present invention) did not exhibit a large variation in the efficiency of cell growth. This indicates that it is not necessary to strictly set culture conditions depending on the kind of cell and the like in Examples.

On the other hand, Comparative Examples using a spinner flask for cultivation widely varied in the efficiency of cell culture depending on the rotational speed of the stirring bar. That is, in Comparative Examples, the state of grown cells greatly varied depending on the rotational speed of the stirring bar irrespective of the kind of cell. This indicates that the optimization of culture conditions is extremely difficult. Further, in Comparative Examples, detachment of the cells from the carriers for cell culture was confirmed.

In this connection, t is to be noted that cell culture was also carried out in the same manner as in each of Examples described above, wherein the cell culture apparatus as shown in FIG. 5, FIG. 7, or FIG. 8 was used and the pattern of a magnetic field was variously changed. The results were the same as those described above.

### Cell Culturing Method of Second Embodiment

1. Preparation of Cell Culture Carriers and Magnetic Particles

(Cell Culture Carriers II-A)

First, 50 g of nylon particles (base bodies) having an average particle size of 150 μm and a density of 1.02 g/cm³, and 0.25 g of hydroxyapatite particles (porous particles obtained by agglomerating primary particles) having a Ca/P ratio of 1.67 and an average particle size of 10 μm were prepared.

The hydroxyapatite particle had a specific surface area of 45 m²/g, and a pore diameter of 600 Å.

Next, these nylon particles and hydroxyapatite particles were fed into a NARA HYBRIDIZATION SYSTEM NHS-1 (manufactured by Nara Machinery Co., Ltd. and having a rated power of 5.5 kW, and a rated current of 23 A), and the system was operated at 6,400 rpm at 32 to 50° C. for 5 minutes. In this way, cell culture carriers II-A coated with hydroxyapatite (each having a structure shown in FIG. 2) were obtained.

The thus obtained cell culture carrier II-A had an average particle size of 151 μm (the average thickness of coating layer of hydroxyapatite was 1 μm) and a density of 1.03 g/cm³.

(Cell Culture Carriers II-B)

Dextran particles having an average particle size of 200 μm and a density of 1.03 g/cm³ (manufactured by Pharmacia) were prepared as cell culture carriers II-B.

(Cell Culture Carriers II-C)

Nylon particles having an average particle size of 150 μm and a density of 1.02 g/cm³ were prepared as cell culture carriers II-C.

(Magnetic Particles II-A)

Ferrite composite nylon particles (each having a structure shown in FIG. 3) having an average particle size of 150 μm and a density of 1.90 g/cm³ were prepared as magnetic particles II-A.

(Magnetic Particles II-B)

First, 50 g of ferrite composite nylon particles having an average particle size of 150 μm and a density of 1.90 g/cm³, and 0.25 g of hydroxyapatite particles (porous particles obtained by agglomerating primary particles) having a Ca/P ratio of 1.67 and an average particle size of 10 μm were prepared.

The hydroxyapatite particle had a specific surface area of 45 m²/g, and a pore diameter of 600 Å.

Next, these ferrite composite nylon particles and hydroxyapatite particles were fed into a NARA HYBRIDIZATION SYSTEM NHS-1 (manufactured by Nara Machinery Co., Ltd. and having a rated power of 5.5 kW, and a rated current of 23 A), and the system was operated at 6,400 rpm at 32 to 50° C. for 5 minutes. In this way, magnetic particles II-B coated with hydroxyapatite were obtained.

The thus obtained magnetic particle II-B had an average particle size of 151 μm (the average thickness of coating layer of hydroxyapatite was 1.0 μm), and a density of 1.93 g/cm³.

2. Culture of Cells

2-1 Culture of Cells Derived from Human Osteosarcoma (HOS)

This cell derived from human osteosarcoma is a cell having a maximum length of about 20 μm.

### Example IIa-1

0.8 g of the cell culture carriers II-A, 0.6 g of the magnetic particles II-A, and 30 mL of a suspension containing 2×10⁵ cells derived from human osteosarcoma (HOS) per milliliter (/mL) were added to 100 mL of Nissui MEM (culture solution). It is to be noted that 10 vol % of bovine fetal serum was added to the Nissui MEM. The volume ratio between the cell culture carriers II-A and the magnetic particles II-A was 70:30.

This culture solution was stored in a culture vessel (which is a heatproof glass jar manufactured by IWAKI-PYREX) of a cell culture apparatus as shown in FIG. 5, and the cells were cultured. As for culture conditions, the pattern of a generated magnetic field was a pattern shown in FIG. 6(a), a pulse interval was 2 seconds, the temperature of the culture solution was 37° C., and a cultivation period was 5 days.

### Example IIa-2

Cell culture was carried out in the same manner as in Example IIa-1 except that the pulse interval was changed to 10 seconds.

### Example IIa-3

Cell culture was carried out in the same manner as in Example I-A except that the magnetic particles II-A were replaced with the magnetic particles II-B. The volume ratio between the cell culture carriers II-A and the magnetic particles II-B was 70:30.

### Example IIa-4

Cell culture was carried out in the same manner as in Example IIa-1 except that 0.8 g of the cell culture carriers II-A was replaced with 0.2 g of the cell culture carriers II-B.

### Comparative Example IIa-1

0.8 g of the carriers for cell culture A and 30 mL of a suspension containing 2×10⁵ cells derived from human osteosarcoma (HOS) per milliliter were added to 100 mL of Nissui MEM (culture solution). It is to be noted that 10 vol % of bovine fetal serum was added to the Nissui MEM.

This culture solution was received in a spinner flask (manufactured by Shibata Scientific Technology Ltd.), and the cells were cultured. Culture conditions were a rotational speed of stirring bar of 30 rpm, a temperature of culture solution of 37° C., and a cultivation period of 5 days.

### Comparative Example IIa-2

Cell culture was carried out in the same manner as in Comparative Example IIa-1 except that the rotational speed of the stirring bar was changed to 60 rpm.

### Comparative Example IIa-3

Cell culture was carried out in the same manner as in Comparative Example IIa-1 except that the cell culture carriers II-A were replaced with the cell culture carriers II-C.

2-2 Culture of Cells Derived from Monkey Kidney (Vero)

This cell derived from a monkey kidney is a cell having a maximum length of about 20 μm.

### Examples IIb-1 to IIb-4 and Comparative Examples IIb-1 to IIb-3

Cell culture was carried out in the same manner as in each of Examples IIa-1 to IIa-4 and Comparative Examples IIa-1 to IIa-3 except that the cells derived from human osteosarcoma were replaced with the cells derived from a monkey kidney.

2-3 Culture of Cells Derived from Mosquito (C6/36)

This cell derived from a mosquito is a cell having a maximum length of about 20 μm.

### Examples IIc-1 to Ic-4 and Comparative Examples IIc-1 to IIc-3

Cell culture was carried out in the same manner as in each of Examples IIa-1 to IIa-4 and Comparative Examples IIa-1 to IIa-3 except that the cells derived from human osteosarcoma were replaced with the cells derived from a mosquito.

3. Evaluation

In each of Examples and Comparative Examples, a predetermined amount of the culture solution was sampled after 3 hours, 1 day, 3 days and 5 days from the beginning of cultivation (beginning of agitation), and then the number of the cells adhering to the surface of the cell culture carriers (15 mg) was counted. The number of the cells was counted by subjecting the cells treated with EDTA or trypsin to trypanblue staining.

The results are shown in Tables 4 to 6.

TABLE 4
(Cells Derived from Human Osteosarcoma (HOS))

| | Cell | | Number of cells (×10⁵ cells/mL) | | | |
|---|---|---|---|---|---|---|
| | Culture | Magnetic | After 3 | After 1 | After 3 | After 5 |
| | Carriers | particles | hours | day | days | days |
| Ex. IIa-1 | II-A | II-A | 0.9 | 4.1 | 9.8 | 10.0 |
| Ex. IIa-2 | II-A | II-A | 1.0 | 4.3 | 9.6 | 9.6 |
| Ex. IIa-3 | II-A | II-B | 1.0 | 4.5 | 10.1 | 11.0 |
| Ex. IIa-4 | II-B | II-A | 0.9 | 4.0 | 9.6 | 9.8 |
| Comp. | II-A | — | 0.9 | 3.5 | 9.0 | 9.0 |
| Ex. A-1 | | | | | | |
| Comp. | II-A | — | 0.8 | 0.9 | 1.2 | 1.0 |
| Ex. IIa-2 | | | | | | |
| Comp. | II-C | — | Measurement | Measurement | Measurement | Measurement |
| Ex. IIa-3 | | | was | was | was | was |
| | | | incapable | incapable | incapable | incapable |

TABLE 5
(Cells Derived from Monkey Kidney (Vero))

| | Carriers | | Number of cells (×10⁵ cells/mL) | | | |
|---|---|---|---|---|---|---|
| | for cell | Magnetic | After 3 | After 1 | After 3 | After 5 |
| | culture | particles | hours | day | days | days |
| Ex. IIb-1 | II-A | II-A | 1.0 | 3.0 | 5.8 | 9.8 |
| Ex. IIb-2 | II-A | II-A | 1.1 | 3.2 | 6.5 | 11.3 |
| Ex. IIb-3 | II-A | II-B | 1.0 | 3.5 | 6.5 | 11.5 |
| Ex. IIb-4 | II-B | II-A | 0.9 | 3.0 | 5.6 | 9.4 |
| Comp. | II-A | — | 0.9 | 2.2 | 4.3 | 4.2 |
| Ex. IIb-1 | | | | | | |
| Comp. | II-A | — | 0.8 | 1.2 | 4.0 | 9.2 |
| Ex. IIb-2 | | | | | | |
| Comp. | II-C | — | Measurement | Measurement | Measurement | Measurement |
| Ex. IIb-3 | | | was | was | was | was |
| | | | incapable | incapable | incapable | incapable |

TABLE 6
(Cells Derived from Mosquito (C6/36))

| | Carriers | | Number of cells (×10⁵ cells/mL) | | | |
|---|---|---|---|---|---|---|
| | for cell | Magnetic | After 3 | After 1 | After 3 | After 5 |
| | culture | particles | hours | day | days | days |
| Ex. IIc-1 | II-A | II-A | 0.8 | 2.2 | 5.3 | 8.0 |
| Ex. IIc-2 | II-A | II-A | 0.8 | 4.2 | 7.5 | 8.9 |
| Ex. IIc-3 | II-A | II-B | 0.9 | 4.2 | 8.0 | 9.2 |
| Ex. IIc-4 | II-B | II-A | 0.8 | 2.1 | 5.5 | 7.9 |
| Comp. | II-A | — | 0.8 | 1.0 | 4.8 | 7.5 |
| Ex. IIc-1 | | | | | | |
| Comp. | II-A | — | 0.7 | 0.9 | 0.9 | 0.8 |
| Ex. IIc-2 | | | | | | |
| Comp. | II-C | — | Measurement | Measurement | Measurement | Measurement |
| Ex. IIc-3 | | | was | was | was | was |
| | | | incapable | incapable | incapable | incapable |

As shown in each of the tables, it has become apparent that all Examples (present invention) exhibit higher efficiency of cell culture as compared with Comparative Examples irrespective of the kind of cell.

Further, even in a case where the pulse interval of generation pattern of a magnetic field was changed, Examples (present invention) did not exhibit a large variation in the efficiency of cell growth. This indicates that in the cases of Examples, it is not necessary to strictly set culture conditions depending on the kind of cell and the like.

On the other hand, Comparative Examples using a spinner flask for cultivation widely varied in the efficiency of cell culture depending on the rotational speed of the stirring bar. That is, in Comparative Examples, the state of grown cells greatly varied depending on the rotational speed of the stirring bar irrespective of the kind of cell. This indicates that the optimization of culture conditions is extremely difficult. Further, in Comparative Examples, detachment of the cells from the carriers for cell culture was confirmed.

It is to be noted that cell culture was carried out in the same manner as in each of Examples described above, wherein the cell culture apparatus as shown in FIG. 5, FIG. 7, or FIG. 8 was used and the pattern of a magnetic field was variously changed. The results were the same as those described above.

### Effect of the Invention

As has been described above, according to the present invention, it is possible to agitate a culture solution uniformly and mildly, thereby enabling cells to grow efficiently.

Further, by appropriately setting the structures of the cell culture carrier and magnetic particle, the effect described above can be further improved.

Finally, it is to be understood that many changes and additions may be made to the embodiments described above without departing from the scope and spirit of the invention as defined in the following claims.

Further, it is also to be understood that the present disclosure relates to subject matter contained in Japanese Patent Applications No. 2003-107143 and No. 2003-107144 (both filed on Apr. 10, 2003) which are expressly incorporated herein by reference in its entirety.

What is claimed is:

1. A method for culturing cells, comprising the steps of:
preparing a cell culture solution containing at least cells to be cultured and granular cell culture carriers to which the cells are allowed to adhere and grow thereon; and
applying a magnetic field to the cell culture solution so as to agitate the cell culture solution by the effect of the magnetic field, whereby the cells adhere to and grow on the surfaces of the cell culture carriers.

2. The method as claimed in claim 1, wherein each of the carriers comprises a magnetic particle having a surface and a coating layer which is provided to cover at least a part of the surface of the magnetic particle so that the cells are allowed to adhere thereto, wherein the cell culture carriers are moved in the culture solution by the application of the magnetic field, thereby agitating the culture solution.

3. The method as claimed in claim 2, wherein the intensity of the magnetic field applied to the cell culture solution is changed with the lapse of time.

4. The method as claimed in claim 2, wherein the position of the magnetic field applied to the cell culture solution is changed with the lapse of time.

5. The method as claimed in claim 2, wherein a density of each of the cell culture carriers is in the range of 0.8 to 2.5 g/cm³.

6. The method as claimed in claim 2, wherein when the average particle size of the cell culture carriers is defined as A μm and the maximum length of the cell allowed to adhere to the cell culture carrier is defined as B μm, A/B is 2 to 100.

7. The method as claimed in claim 2, wherein the average particle size of the cell culture carriers is in the rage of 50 to 500 μm.

8. The method as claimed in claim 2, wherein the coating layer is mainly made of a calcium phosphate-based compound.

9. The method as claimed in claim 8, wherein the coating layer is formed from fine particles of the calcium phosphate-based compound wherein the particles being partially embedded in a surface area including and adjacent to the surface of the magnetic particle.

10. The method as claimed in claim 9, wherein the particles of the calcium phosphate-based compound are formed from porous particles, and the coating layer is formed by colliding the porous particles to the surface of the magnetic particle.

11. The method as claimed in claim 2, wherein each of the magnetic particles is formed by compounding a resin material and a magnetic material.

12. The method as claimed in claim 1, wherein the culture solution further contains magnetic particles, and the magnetic particles are moved in the culture solution by the application of the magnetic field, thereby agitating the culture solution.

13. The method as claimed in claim 12, wherein the intensity of the magnetic field applied to the cell culture solution is changed with the lapse of time.

14. The method as claimed in claim 12, wherein the position of the magnetic field applied to the cell culture solution is changed with the lapse of time.

15. The method as claimed in claim 12, wherein each of the carriers comprises a base body made of a resin material and having a surface and a coating layer which is provided to cover at least a part of the surface of the base body so that the cells are allowed to adhere thereto.

16. The method as claimed in claim 15, wherein the coating layer is mainly made of a calcium phosphate-based compound.

17. The method as claimed in claim 16, wherein the coating layer is formed from fine particles of the calcium phosphate-based compound wherein the particles being partially embedded in a surface area including and adjacent to the surface of the base body.

18. The method as claimed in claim 17, wherein the particles of the calcium phosphate-based compound are formed from porous particles, and the coating layer is formed by colliding the porous particles to the surface of the magnetic particle.

19. The method as claimed in claim 12, wherein a density of each of the cell culture carriers is in the range of 0.8 to 1.4 g/cm³.

20. The method as claimed in claim 12, wherein when the average particle size of the cell culture carriers is defined as A μm and the maximum length of the cell that is allowed to adhere to the cell culture carrier is defined as B μm, A/B is 2 to 100.

21. The method as claimed in claim 12, wherein when the average particle size of the cell culture carriers is defined as A μm and the average particle size of the magnetic particles is defined as C μm, C/A is 0.02 to 10.

22. The method as claimed in claim 12, wherein the average particle size of the cell culture carriers is in the rage of 50 to 500 μm.

23. The method as claimed in claim 12, wherein each of the magnetic particles is formed by compounding a resin material and a magnetic material.

24. The method as claimed in claim 12, wherein a density of each of the magnetic particles is in the range of 0.8 to 2.5 g/cm³.

25. The method as claimed in claim 12, wherein the average particle size of the magnetic particles is in the range of 10 to 500 μm.

26. The method as claimed in claim 12, wherein each of the magnetic particles further comprises a coating layer which covers at least a part of the surface of the magnetic powder so that the cells are allowed to adhere thereto.

27. The method as claimed in claim 26, wherein the coating layer is mainly made of a calcium phosphate-based compound.

28. The method as claimed in claim 27, wherein the coating layer is formed from fine particles of the calcium phosphate-based compound wherein the particles being partially embedded in a surface area including and adjacent to the surface of the magnetic particle.

29. The method as claimed in claim 28, wherein the particles of the calcium phosphate-based compound are formed from porous particles, and the coating layer is formed by colliding the porous particles to the surface of the magnetic particle.

30. The method as claimed in claim 12, wherein a mixing ratio of the magnetic particles and the cell culture carriers is in the range of 10:90 to 50:50 in a volume ratio.

31. Cell culture carriers to which cells are allowed to adhere to and grow on surfaces thereof, wherein each of the carriers comprising:
a magnetic particle having a surface;
a coating layer which is provided to cover at least a part of the surface of the magnetic particle so that the cells are allowed to adhere thereto.

32. The cell culture carriers as claimed in claim 31, wherein a density of the carrier is in the range of 0.8 to 2.5 g/cm³.

33. The cell culture carriers as claimed in claim 31, wherein when the average particle size of the cell culture carriers is defined as A μm and the maximum length of the cell that is allowed to adhere to the cell culture carrier is defined as B μm, A/B is 2 to 100.

34. The cell culture carriers as claimed in claim 31, wherein the particle size of the cell culture carriers is in the rage of 50 to 500 μm.

35. The cell culture carriers as claimed in claim 31, wherein the coating layer is mainly made of a calcium phosphate-based compound.

36. The cell culture carriers as claimed in claim 35, wherein the coating layer is formed from fine particles of the calcium phosphate-based compound wherein the fine particles being partially embedded into the magnetic particle at the vicinity of the surface thereof.

37. The cell culture carriers as claimed in claim 36, wherein the fine particles of the calcium phosphate-based compound are formed from porous particles, and the coating layer is formed by colliding the porous particles to the surface of the magnetic particle.

38. The cell culture carriers as claimed in claim 31, wherein the magnetic particles are formed by compounding a resin material and a magnetic material.

39. A cell culture apparatus, comprising:
a cell culture vessel for storing a cell culture solution containing at least cells to be cultured and granular cell culture carriers to which the cells are allowed to adhere and grow thereon; and
at least one magnetic field generator for applying a magnetic field to the culture solution to agitate the culture solution by the effect of the magnetic field.

40. The cell culture apparatus as claimed in claim 39, wherein each of the carriers comprises a magnetic particle having a surface and a coating layer which is provided to cover at least a part of the surface of the magnetic particle so that the cells are allowed to adhere thereto, wherein the cell culture carriers are moved in the culture solution by the application of the magnetic field, thereby agitating the culture solution.

41. The cell culture apparatus as claimed in claim 39, wherein the culture solution further contains magnetic particles, and the magnetic particles are moved in the culture solution by the application of the magnetic field, thereby agitating the culture solution.

42. The cell culture apparatus as claimed in claim 39, wherein the magnetic field generator is constructed so that the intensity of the generated magnetic field is changed with the lapse of time.

43. The cell culture apparatus as claimed in claim 39, wherein the magnetic field generator is constructed so that the position of the generated magnetic field is changed with the lapse of time.

44. The cell culture apparatus as claimed in claim 39, wherein the magnetic field generator is arranged around the outer periphery of the cell culture vessel.

45. The cell culture apparatus as claimed in claim 39, wherein the magnetic field generator is provided so as to come into contact with the culture solution.

46. The cell culture apparatus as claimed in claim 39, wherein the magnetic field generator is arrange in the vicinity of the liquid surface of the culture solution contained in the cell culture vessel.

47. The cell culture apparatus as claimed in claim 39, wherein the at least one magnetic field generator includes two or more magnetic field generators.
